(19) United States
(12) Patent Application Publication
(10) Pub. No.: US 2023/0065361 A1
(43) Pub. Date: Mar. 2, 2023

(54) METHOD FOR INCREASING USE EFFICIENCY OF LIPASE DURING ENZYMATIC REACTION

(71) Applicant: Jiangnan University, Wuxi City (CN)

(72) Inventors: Xiaosan WANG, Wuxi City (CN); Ye HUANG, Wuxi City (CN); Zhuoneng HUANG, Wuxi City (CN); Ye CHEN, Wuxi City (CN); Keying LIU, Wuxi City (CN); Yaqi HUANG, Wuxi City (CN); Xiaohan WANG, Wuxi City (CN); Ming CHANG, Wuxi City (CN)

(21) Appl. No.: 17/876,515

(22) Filed: Jul. 28, 2022

(30) Foreign Application Priority Data
Aug. 18, 2021 (CN) 202110949528.3

(51) Int. Cl. C12P 7/6454

(52) U.S. Cl. CPC C12P 7/6454

(57) ABSTRACT

The present disclosure discloses a method for increasing the usage efficiency of a lipase in the enzymatic reaction, belonging to the technical field of food processing. In the present disclosure, the peroxide value of raw materials required for enzymatic synthesis of a structured lipid is reduced by a distillation method or an adsorption method, and then the raw material and a lipase undergo catalytic reaction to synthesize the structured lipid, so as to maintain the activity of the lipase and increase the reuse rate of the enzyme. The distillation method includes deodorization and molecular distillation. The present disclosure provides a method for manufacturing a structured lipid by a high efficiency and low-cost enzymatic synthesis, addressing to the issues such as large enzyme consumption, expansive cost, low enzyme utilization rate in the traditional industrial production. Therefore, the method provided by the present disclosure has certain development potential and practical significance.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

The present disclosure belongs to the technical field of food processing, and particularly relates to a method for increasing the use efficiency of a lipase during enzymatic reaction.

### BACKGROUND OF THE INVENTION

Fat is an essential nutrient for a human body and widely exists in many kinds of food. The main component of fat is triacylglycerol formed by esterification of glycerol and different fatty acids. In recent years, researchers have studied on medium carbon-chain fatty acid triacylglycerols and long carbon-chain fatty acid triacylglycerols. In order to integrate the advantages of both, structured lipids have appeared. Currently, the researches of structured lipids are mainly focusing on human milk fat substitutes, cocoa butter replacers, medium and long carbon-chain fatty acid structured lipids, triacylglycerols containing polyunsaturated fatty acids, etc. The human milk fat substitutes, such as OPO and OPL, added to infant formula milk powder can effectively simulate the composition of human milk fat and are beneficial for lipid absorption, maintaining the balance of mineral content and reducing constipation. Cocoa butter replacer is another kind of structured lipids in high demand, which can relieve the resource shortage of cocoa butter raw materials. The medium and long carbon-chain fatty acid structured lipids integrate the advantages of rapid metabolism and quick energy supply of medium carbon-chain fatty acid triacylglycerols and high nutrition of long carbon-chain fatty acid triacylglycerols, which are greatly beneficial to human bodies. The triacylglycerols containing polyunsaturated fatty acids can effectively provide human body with essential fatty acids and reduce blood lipid.

At present, an enzymatic synthesis method is the most widely used method for synthesizing structured lipids in industrial production. Compared with the traditional inorganic or organic enzymatic synthesis method, lipase-catalyzed structured lipids synthesis method has the advantages of moderate conditions, high catalytic efficiency, few byproducts and high selectivity. However, lipases are high in price. Some immobilized lipases such as Novozym 435, Lipozyme TL IM and LipozymeRM IM are frequently applied to processing and production of human milk fat substitutes, but these enzymes are generally very expansive. Therefore, the industry has been looking for ways to reuse lipase to increase the utilization rate of enzymes and reduce the cost, for example, an electric field method, an organic solvent method and a mixed oil soaking method, but these methods are disadvantageous for continuous production, because the enzyme needs to be adjusted after each treatment which is cumbersome, not environmental-friendly and low in efficiency.

### SUMMARY OF THE INVENTION

### Technical Problems

The existing lipases used for synthesizing structured lipids have high cost and low reusability.

### Technical Solution

In order to solve the above problems, the present invention retains the activity of lipase by reducing a peroxide value of a raw material required for synthesizing a structured lipid, so that the reuse times of the enzyme are increased. Thus, the present disclosure provides a method for enzymatic synthesis of a structured lipid in high efficiency and low cost, which is of practical significance.

Specifically, the present disclosure provides the following technical solutions: a method for increasing the use efficiency of a lipase during an enzymatic reaction, including conducting a peroxide value reduction treatment on a substrate used for synthesizing a structured lipid, and subjecting the substrate to undergo a catalytic reaction with a lipase to synthesize the structured lipid, wherein the peroxide value reduction treatment includes a distillation method and an adsorption method.

In one embodiment, the distillation method includes deodorization and molecular distillation.

In one embodiment, the temperature of the deodorization is 220° C.-270° C., preferably 240° C.-270° C.

In another embodiment, the time of the deodorization is 30 mins-150 mins, and an operation pressure is less than 8 mbar.

In one embodiment, the temperature of the evaporation surface of the molecular distillation is 200° C.-290° C., preferably 220° C.-250° C., and the operation pressure is less than 10 mbar.

In another embodiment, an adsorbent used in the adsorption method includes silica gel, activated clay, activated carbon, zeolite, diatomite, attapulgite or any combination thereof; more preferably silica gel or activated carbon.

In one embodiment, the addition amount of the adsorbent is 0.5%-6% of the substrate weight, preferably 1%-3%.

In another embodiment, the adsorption time of the adsorption method is less than 60 mins, preferably less than 30 mins.

In some embodiments, the lipase includes any lipase used for catalyzing hydrolysis reaction, ester exchange reaction or esterification reaction.

In one embodiment, the lipase used for catalyzing hydrolysis reaction includes, but not limited to, one or more of lipases derived from Candida cylindracea, Pseudomonas fluorescens, Rhizopusoryzea and Pseudomonas cepacia.

In one embodiment, the lipase used for catalyzing ester exchange reaction includes, but not limited to, one or more of lipases derived from Candida Antarctica, Thermomyceslanuginosus, Rhizopusoryzea and Rhizomucormiehei.

In another embodiment, the lipase used for catalyzing esterification reaction includes, but not limited to, one or more of lipases derived from Rhizomucormiehei, Thermomyceslanuginosus, Burholderiacepacia, Candida Antarctica, Rhizopusoryzea, Pseudomonas cepacia and Asperigillusniger.

In one embodiment, the conditions for catalyzing the reaction can be identical with the catalytic conditions disclosed in the prior art.

In one embodiment, the substrates of the ester exchange reaction and the hydrolysis reaction may be an oil or a fractional extract thereof.

In some embodiments, the substrate includes plant oil, animal oil, microbial oil or any combination thereof.

In one embodiment, the plant oil is selected from, but not limited to, soybean oil, peanut oil, castor oil, high oleic sunflower oil, olive oil, coconut oil, flaxseed oil, palm stearin, rapeseed oil or any combination thereof.

In one embodiment, the animal oil is selected from, but not limited to, lard, pomfret oil, tilapia oil, Pasha fish oil or any combination thereof.

In one embodiment, the microbial oil is selected from, but not limited to, ARA-rich oils, DHA-rich oils or any combination thereof.

In another embodiment, the substrate of the esterification reaction is an acyl donor including, but not limited to, oleic acid, linoleic acid and other acyl donors.

In one embodiment, the catalytic ester exchange reaction includes an acidolysis reaction, an alcoholysis reaction and an ester-ester exchange reaction.

In one embodiment, the structured lipid includes, but not limited to, a human milk fat substitute, a cocoa butter replacer, a medium and long carbon-chain fatty acid structured lipid and a triacylglycerol containing a polyunsaturated fatty acid.

The present disclosure also provides a use of the above method in the fields of structured lipid preparation and food.

The present disclosure has the beneficial effects:

In the present disclosure, the raw material required for enzymatic synthesis of a structured lipid is subjected to a peroxide value reduction treatment, so that the activity of lipase can be better maintained during the enzymatic synthesis of the structured lipid. The reuse times of the lipase are effectively increased, improving the utilization rate of the lipase. Therefore, the problems of high lipase cost and low utilization rate in the process of industrial production are effectively solved. In other words, the method of the present disclosure is significant for the industry.

### BRIEF DESCRIPTION OF THE DRAWINGS

FIGS. 1A-1B shows reuse times of a lipase with or without peroxide value reduction treatment of a raw material, and FIG. 1A represents reuse times of the lipase under the condition of comparative example 1 and FIG. 1B represents reuse times of the lipase under the condition of example 1;

FIGS. 2A-2B are liquid chromatograms (differential detector) of 2-monoglycerides obtained after a lipase with or without peroxide value reduction treatment of a raw material is used for 10 times in alcoholysis reaction, and FIG. 2A represents a liquid chromatogram of an alcoholysis crude product under the condition of comparative example 2 and FIG. 2B represents a liquid chromatogram of an alcoholysis crude product under a deodorization condition in example 2; and

FIGS. 3A-3B are liquid chromatograms (differential detector) of diglycerides obtained after a lipase with or without peroxide value reduction treatment of a raw material is used for 10 times in hydrolysis reaction, and FIG. 3A represents a liquid chromatogram of a hydrolysis crude product under the condition of comparative example 3 and FIG. 3B represents a liquid chromatogram of a hydrolysis crude product under a deodorization condition in example 4.

### DETAILED DESCRIPTION

Next, the present disclosure will be further described in combination with examples, but the embodiments of the present disclosure are not limited thereto.

For more clearly and easily understanding the above objective, features and advantages of the present disclosure, the specific embodiments of the present disclosure will be illustrated in detail in combination with specific examples below.

The following descriptions set forth many specific details so as to sufficiently understand the present disclosure, but the present disclosure can also be implemented by using other manners different from the descriptions, and similar popularizations can be made by those skilled in the art without departing from the connotation of the present disclosure. Thus, the present disclosure is not limited by the following disclosed specific examples.

Then, “one embodiment” or “embodiment” described herein refers to including specific features, structures or characteristics of at least one implementation mode of the present disclosure. In this specification, “in one embodiment” occurring in different places does not refer to the same embodiment or embodiments repellent to other embodiments alone or selectively.

1. A structured lipid analysis method:

\textcircled{1} Operation and parameters for HPLC-ELSD detection: referring to a method by Liang Gao etc. (Liang Gao, Xuwei Yu, Feng Zou, etc., RESEARCH ON ENZYMATIC SYNTHESIS OF STRUCTURED LIPID OF 1-OLEIC ACID-2-PALMITIC ACID-3-LINOLEIC TRIGLYCERIDE [J]. Chinese Oil, 2020, 45(08):66-70), the TAG compositions in oil samples are detected and analyzed by a reversed phase high performance liquid chromatograph (RP-HPLC) with an evaporation photodetector. 20 g of sample is taken and dissolved into 1.0 mL of chromatographic level n-hexane. After film filtration, it is analyzed by HPLC. Conditions for HPLC are as follows: Lichrospher C18 chromatographic column (250 mm×4.6 mm×5 μm); the temperature of the evaporation photodetector is 55° C.; an air flow rate is set as 1.8 mL/min, a gain value is 1; an eluting flow rate is 0.8 mL/min; a sampling concentration is 20 mg/mL, and a sampling volume is 20 μL.

Procedures for HPLC: elution procedures of a mobile phase are shown in Table 1, and quantitative analysis is performed in combination with a peak area normalization method.

TABLE 1
Elution procedures of a mobile phase via HPLC

| Time/min | Acetonitrile/% | Isopropanol/% |
|---|---|---|
| 0 | 60 | 40 |
| 40 | 60 | 40 |
| 45 | 55 | 45 |
| 85 | 55 | 45 |
| 90 | 60 | 40 |

\textcircled{2} Operation and parameters for HPLC-RID analysis: referring to a method by Wang etc. (Wang W F, Li, T, Qin, X L, et al. Production of lipase SMG1 and its application in synthesizing diacylglyecrol [J]. Journal of Molecular Catalysis B-Enzymatic, 2012, 77(6): 87-91.). Quantitative analysis of lipid components is performed on the post-reaction system by HPLC-RID. Chromatographic conditions are as follows: chromatographic column Sepax HP-Silica (4.6 mm×250 mm×5 μm), and the temperature of the column is 30° C.; the concentration of a sample is 10 mg/mL, and a sampling volume is 15 μL; a ratio of mobile phase n-hexane: isopropanol: formic acid is 15:1:0.03 (v/v/v), and a flow rate is 1 mL/min. Each lipid component is qualified through standards, the sample concentration and a peak area are in a linear relationship, and relative compositions of each substance are represented through an area normalization method (%).

2. Peroxide value analysis method: peroxide value is determined by reference with “Determination of Peroxide value in Food of GB5009.227-2016 National Food Safety Standard”.

3. Determination of enzyme activity: based on the content of C52 triacylglycerol (or diglyceride structured lipid, or monoglyceride structured lipid) in a product as an ordinate, the remaining 80% enzyme activity is 80%×the content of C52 triglyceride (or diglyceride structured lipid, or monoglyceride structured lipid) obtained by first enzymatic reaction.

### EXAMPLE 1: (ESTER EXCHANGE-PREPARATION OF OPO VIA ACIDOLYSIS REACTION)

Peroxide value reduction treatment: a palm stearin raw material with a peroxide value of 7.4 mmol/kg is subjected to a peroxide value reduction treatment. The treatment method is shown in Table 2.

Synthesis of a structured lipid through ester exchange: the palm stearin treated with the peroxide value reduction treatment and an oleic acid (a molar ratio of 1:10) are mixed in a 25 mL reactor. When the reaction substrates are dissolved and stabilized, LipozymeRM IM (derived from Rhizomucormiehei) whose weight is 10% of the total weight of the raw material system is added, and the above materials are reacted for 6 h at 60° C. After the reaction, the reaction product is centrifuged for 3 min at 4000 r/min to retrieve the lipase and the free lipases are removed for obtaining an abundant amount of C52 structured lipid. The content of the C52 triacylglycerol is evaluated by HPLC-ELSD, i.e., the content of the C52 triacylglycerol obtained by the first enzymatic reaction. The retrieved lipase is added into the next reaction of structured lipid enzymatic synthesis with the identical conditions of the first reaction. The lipase is reused for conducting the reaction repeatedly until the remaining enzyme activity of the lipase is down to 80%, and the reuse times of the lipase are calculated.

The influences of different peroxide value treatments with different conditions on reuse times of enzyme are investigated: raw materials are treated respectively with molecular distillation, deodorization and adsorption methods, the reuse times of the enzyme under different treatment conditions are measured, see Table 2. The raw materials used in control group have the same initial peroxide values without any peroxide value reduction treatments.

TABLE 2
Reuse times of enzyme used in example 1 in acidolysis
reactions (the reaction substrate is treated with
or without peroxide value reduction treatment)

| Treatment | | | Constant | | Reuse times |
|---|---|---|---|---|---|
| mode | Variable | | conditions | | of enzymes |
| Molecular | 200° | C. | 6 | mbar | 14 |
| distillation | 230° | C. | | | 16 |
| | 250° | C. | | | 14 |
| | 280° | C. | | | 15 |
| | 4 | mbar | 230° | C. | 16 |

| | 6 | mbar | | 16 |
|---|---|---|---|---|
| | 8 | mbar | | 15 |
| | 10 | mbar | | 14 |
| Deodorization | 220° | C. | 4 mbar, and 90 min | 13 |
| | 250° | C. | | 17 |
| | 270° | C. | | 16 |
| | 30 | min | 4 mbar, and 250° C. | 14 |
| | 60 | min | | 15 |
| | 90 | min | | 17 |
| | 150 | min | | 17 |
| | 4 | mbar | 90 min, and 250° C. | 17 |
| | 6 | mbar | | 16 |
| | 8 | mbar | | 16 |
| Adsorption | Activated clay | | 20 min, room | 13 |
| | Activated carbon | | temperature, and 2% | 17 |
| | Silica gel | | adsorbent addition | 18 |
| | Zeolite | | amount | 14 |
| | Kieselguhr | | | 14 |
| | Attapulgite | | | 13 |
| | 1% silica gel | | 20 min, room | 17 |
| | 2% silica gel | | temperature | 18 |
| | 3% silica gel | | | 17 |
| | 10 | min | 2% silica gel, and | 16 |
| | 20 | min | room temperature | 17 |
| | 30 | min | | 17 |

| | | | |
|---|---|---|---|
| No (control | No | No | 11 |
| group) | | | |

As shown in Table 2, distillation and evaporation methods both can effectively improve the reuse times of the enzyme and increase the utilization rate of the enzyme. Furthermore, the adsorption method shows the most significant effect on improving reuse times, especially with using silica gel and activated carbon for adsorption.

### EXAMPLE 2: (ESTER EXCHANGE-PREPARATION OF 2-MONOGLYCERIDE VIA ALCOHOLYSIS REACTION)

Peroxide value reduction treatment: a high oleic sunflower oil raw material with a peroxide value of 8.2 mmol/kg is subjected to peroxide value reduction treatment (see Table 3).

Synthesis of a structured lipid through ester exchange: the treated high oleic sunflower oil and ethanol (a molar ratio of 1:10) are mixed in a 25 mL reactor. When the reaction substrates are dissolved and stabilized, Lipozyme TL IM (derived from sparsely spongy-like Thermomyceslanuginosus) whose weight is 10% of the total weight of the raw material system is added, and the above materials are reacted for 5 h at 30° C. After the reaction, the reaction product is centrifuged for 3 min at 4000 r/min to retrieve the lipase and free lipases are removed for obtaining a certain amount of 2-monoglyceride (2-MAG). The content of 2-MAG is evaluated by HPLC-RID, i.e., the content of 2-MAG obtained by the first enzymatic reaction. The retrieved lipase is added into the next reaction of structured lipid enzymatic synthesis with the identical conditions of the first reaction. The lipase is reused for conducting the reaction repeatedly until the remaining enzyme activity of the lipase is down to 80%, and the reuse times of the lipase are calculated. Reuse times of the enzyme under different conditions are shown in Table 3.

TABLE 3
Reuse times of the enzyme used in example 2 in alcoholysis
reaction (a reaction substrate is treated with or
without peroxide value reduction treatment)

| Treatment | | | Constant | | Reuse times |
|---|---|---|---|---|---|
| mode | Variable | | conditions | | of enzymes |
| Molecular | 200° | C. | 6 | mbar | 11 |
| distillation | 230° | C. | | | 13 |
| | 250° | C. | | | 12 |
| | 280° | C. | | | 13 |
| | 4 | mbar | 230° | C. | 13 |

| | 6 | mbar | | 13 |
|---|---|---|---|---|
| | 8 | mbar | | 12 |
| | 10 | mbar | | 12 |
| Deodorization | 220° | C. | 4 mbar, and 90 min | 12 |
| | 250° | C. | | 14 |
| | 270° | C. | | 13 |
| | 30 | min | 4 mbar, and 250° C. | 11 |
| | 60 | min | | 12 |
| | 90 | min | | 14 |
| | 150 | min | | 12 |
| | 4 | mbar | 90 min, and 250° C. | 14 |
| | 6 | mbar | | 12 |
| | 8 | mbar | | 12 |
| Adsorption | Activated clay | | 20 min, room | 11 |
| | Activated carbon | | temperature, and 2% | 13 |
| | Silica gel | | adsorbent addition | 14 |
| | Zeolite | | amount | 10 |
| | kieselguhr | | | 9 |
| | attapulgite | | | 10 |
| | 1% silica gel | | 20 min, and room | 13 |
| | 2% silica gel | | temperature | 14 |
| | 3% silica gel | | | 13 |
| | 10 | min | 2% silica gel, and | 11 |
| | 20 | min | room temperature | 14 |
| | 30 | min | | 12 |

| | | | |
|---|---|---|---|
| No (control | No | No | 8 |
| group) | | | |

### EXAMPLE 3: (ESTERIFICATION REACTION-PREPARATION OF TRIGLYCERIDE)

Peroxide value reduction treatment: an oleic acid with a peroxide value of 7.8 mmol/kg is subjected to peroxide value reduction treatment (see Table 4).

Synthesis of a structured lipid through esterification: the treated oleic acid and glycerinum (a molar ratio of 3:1) and Lipozyme 435 (derived from Candida antarctica) whose weight is 10% of the weight of the system are reacted together to synthesize the structured lipid, and the reacting temperature is 55° C. and the reaction time is 6 h. After the reaction, the lipase is retrieved and a rich amount of triolein triglyceride (OOO) structured lipid is obtained. The content of OOO is evaluated by HPLC-ELSD, namely, the content of OOO obtained by first enzymatic reaction. The retrieved lipase is added into the next structured lipid synthesis reaction with the identical conditions of the first reaction. The lipase is reused for conducting the reaction repeatedly until the remaining enzyme activity of the lipase is down to 80%, and the reuse time of the lipase is calculated. Reuse times of the enzyme under different conditions are shown in Table 4.

TABLE 4
Reuse times of an enzyme in example 2 in esterification
reaction (a reaction substrate is treated with or
without to peroxide value reduction treatment)

| Treatment | | Constant | Reuse times |
|---|---|---|---|
| mode | Variable | conditions | of enzymes |
| Adsorption | Activated clay | 20 min, room | 16 |
| | Activated carbon | temperature, and 2% | 15 |
| | Silica gel | adsorbent addition | 17 |
| | Zeolite | amount | 15 |
| | Kieselguhr | | 15 |
| | attapulgite | | 15 |
| | 1% silica gel | 20 min, and room | 15 |
| | 2% silica gel | temperature | 17 |
| | 3% silica gel | | 15 |
| | 10 min | 2% silica gel, and | 15 |
| | 20 min | room temperature | 17 |
| | 30 min | | 16 |
| No (control | No | No | 12 |
| group) | | | |

### EXAMPLE 4: (HYDROLYSIS REACTION-PREPARATION OF DIGLYCERIDE)

Peroxide value reduction treatment: a soybean oil raw material with a peroxide value of 9.3 mmol/kg is subjected to peroxide value reduction treatment (see Table 5).

Production of a structured lipid through hydrolysis: the treated soybean oil raw material and water (a molar ratio was 1:1) are reacted with Lipase DF IM lipase (derived from Rhizopusoryzae) whose weight is 10% of the weight of the system, and the reaction is carried out under the condition of stirring with the reaction temperature of 35° C., and the hydrolysis time is 1 h. After the reaction, the supernatant is centrifuged for 15 min at 8000 r/min to obtain diglyceride (DAG) and retrieve the lipase The content of DAG is evaluated by HPLC-RID, namely, the content of DAG obtained by the first enzymatic reaction. The retrieved lipase is added into the next structured lipid synthesis reaction with the identical conditions of the first reaction. The lipase is reused for conducting the reaction repeatedly until the remaining enzyme activity of the lipase is down to 80%, and the reuse times of the enzyme are calculated. Reuse times of the enzyme under the different conditions are shown in Table 5.

TABLE 5
Reuse times of enzyme in example 4 in hydrolysis
reaction (a reaction substrate is treated with
or without peroxide value reduction treatment)

| Treatment | | | Constant | | Reuse times |
|---|---|---|---|---|---|
| mode | Variable | | conditions | | of enzymes |
| Molecular | 200° | C. | 10 | mbar | 15 |
| distillation | 230° | C. | | | 17 |
| | 250° | C. | | | 16 |
| | 280° | C. | | | 18 |
| | 4 | mbar | 280° | C. | 16 |

| | 6 | mbar | | 17 |
|---|---|---|---|---|
| | 8 | mbar | | 17 |
| | 10 | mbar | | 18 |
| Deodorization | 220° | C. | 4 mbar, and 150 min | 16 |
| | 250° | C. | | 19 |
| | 270° | C. | | 18 |
| | 30 | min | 4 mbar, and 250° C. | 15 |
| | 60 | min | | 16 |
| | 90 | min | | 18 |
| | 150 | min | | 19 |
| | 4 | mbar | 150 min, and 250° C. | 19 |
| | 6 | mbar | | 17 |
| | 8 | mbar | | 16 |
| Adsorption | Activated clay | | 20 min, room | 16 |
| | Activated carbon | | temperature, and 2% | 18 |
| | Silica gel | | adsorbent addition | 19 |
| | Zeolite | | amount | 15 |
| | kieselguhr | | | 15 |
| | Attapulgite | | | 14 |
| | 1% silica gel | | 20 min, and room | 19 |
| | 2% silica gel | | temperature | 19 |
| | 3% silica gel | | | 18 |
| | 10 | min | 2% silica gel, and | 16 |
| | 20 | min | room temperature | 19 |
| | 30 | min | | 17 |

| | | | |
|---|---|---|---|
| No (control | No | No | 14 |
| group) | | | |

### EXAMPLE 5: (OPTIMIZATION OF ENZYME)

Peroxide value reduction treatment: a palm stearin raw material with a peroxide value of 7.4 mmol/kg is deodorized for 90 min under 4 mbar at 250° C. (see Table 5).

Synthesis of a structured lipid: the treated palm stearin raw material and an oleic acid (a molar ratio was 1:10) are mixed in a 25 mL reactor. When the reaction substrate is dissolved and stabilized, a lipase whose weight is 10% of the total weight of the raw material system is added for catalyzing ester exchange reaction, and the reaction is carried out for 6 h at 60° C. After the reaction, the obtained product is centrifuged for 3 min at 4000 r/min to retrieve the lipase and remove the free fatty acid for obtaining a rich amount of C52 structure lipid. The content of C52 triglyceride is evaluated by HPLC-RID, namely, the content of the C52 triglyceride obtained by the first enzymatic reaction. The retrieved lipase is added into the next structured lipid enzymatic synthesis reaction with the identical conditions of the first reaction. The lipase is reused for conducting the reaction repeatedly until the remaining enzyme activity of the lipase is down to 80%, and the reuse times of the enzyme is calculated.

Influences of different lipases on reuse times are investigated: different lipases are respectively used for reaction, the rest conditions remain the same, and the reuse times of different lipases are respectively measured, see Table 6.

TABLE 6
Reuse times of different lipases obtained
in example 5 and participating in reaction

| | | Reuse times | |
|---|---|---|---|
| | | of enzymes when | |
| | | raw materials | Reuse times |
| | | are subjected to | of enzymes |
| | | molecular | when raw |
| Names and sources | | distillation | materials are |
| of enzymes | Type | treatment | not treated |
| LipozymeRM IM | Immo- | 18 | 14 |
| (derived from | bilized | | |
| Rhizomucormiehei) | enzymes | | |
| LipozymeTL IM | | 8 | 3 |
| (derived from | | | |
| Thermomyceslanuginosus) | | | |
| Lipase CL“Amano”IM | | 16 | 12 |
| (derived from Candida | | | |
| Antarctica) | | | |
| Novozym 435 | | 15 | 12 |
| (derived from | | | |
| Candidaantarctica B) | | | |
| Lipozyme 435 | | 16 | 13 |
| (derived from | | | |
| Candidaantarctica) | | | |
| NS40086 (derived from | | 17 | 13 |
| Rhizomucormiehei) | | | |
| Lipase DFIM | Free | 15 | 10 |
| (derived from | enzymes | | |
| Rhizopusoryzae) | | | |
| Lipase PS | | 7 | 4 |
| (derived from | | | |
| Burholderiacepacia) | | | |
| Lipase A-12 | | 6 | 4 |
| (derived from | | | |
| Asperigillusniger) | | | |
| Lipase MER | | 5 | 3 |
| (derived from | | | |
| Rhizopusoryzae) | | | |
| Lipase M-10SD | | 5 | 3 |
| (derived from | | | |
| Rhizomucorjavanicus) | | | |

### Comparative Example 1: (Compared With Example 1)

Different from example 1, raw materials used in comparative example 1 are not subjected to peroxide value reduction treatment. Experimental conditions and steps for synthesizing the structured lipid through ester exchange acidolysis are identical to example 1.

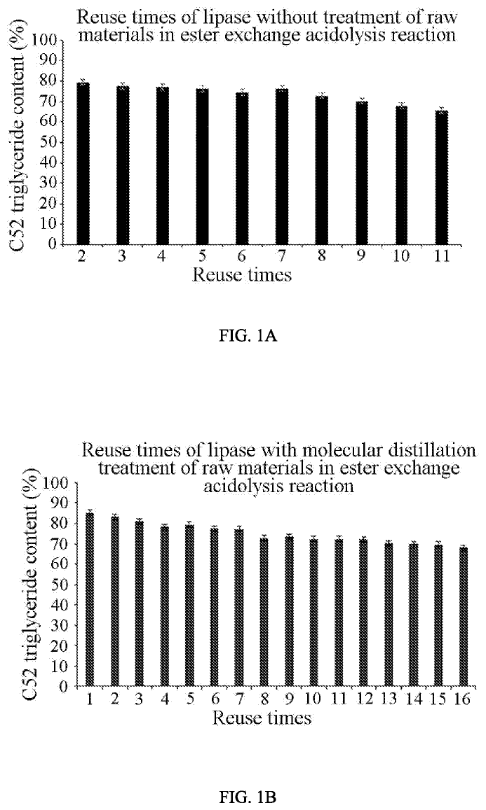

The enzyme used after the molecular distillation of the raw materials under the optimal conditions (6 mbar, and 230° C.) in example 1 is taken for comparison, as shown in FIGS. 1A-1B. FIG. 1A shows the reuse times of the lipase and the content of the product in an ester exchange acidolysis reaction when the raw materials are not treated with any treatment, and FIG. 1B shows the reuse times of the lipase and the content of the product in the ester exchange acidolysis reaction when the raw materials are subjected to molecular distillation treatment. It is indicated that reduction in the peroxide values of the raw materials is extremely beneficial for maintaining the activity of the enzyme and increasing the reuse times of the enzyme.

### Comparative Example 2: (Compared With Example 2)

Different from example 2, raw materials used in comparative example 2 are not subjected to peroxide value reduction treatment. Experimental conditions and steps for synthesizing the structured lipid through ester exchange alcoholysis are identical to example 2.

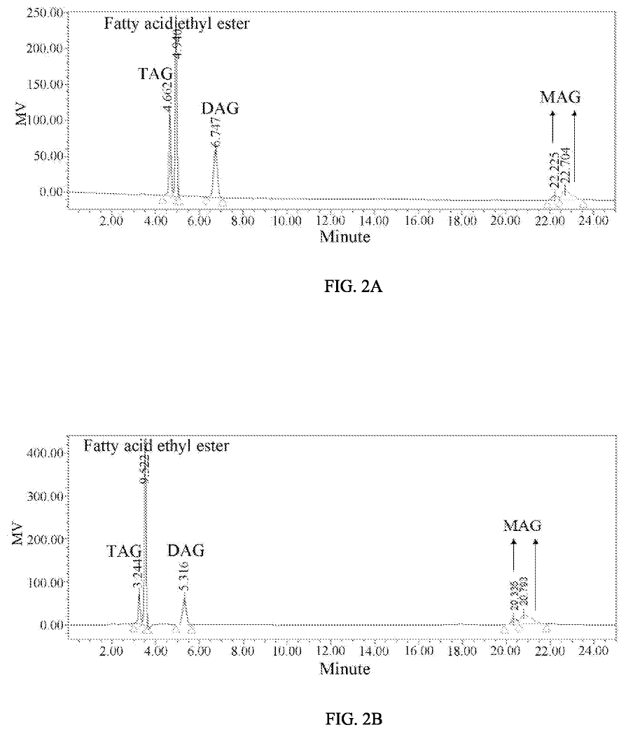

The enzyme retrieved after the alcoholysis reaction of the raw materials in example 2 treated with deodorization under the optimal conditions (4 mbar, 90 min, and 250° C.) is taken and used for synthesis reaction for 10 times, and the content of 2-MAG obtained from 10 times reaction is compared with the content of 2-MAG obtained from 10 times reaction using the enzyme of comparative example 2, see FIG. 2A-2B. FIG. 2A is a liquid chromatogram of raw materials with no peroxide value reduction treatment, and FIG. 2B is a liquid chromatogram of the raw materials subjected to deodorization treatment. In FIG. 2A, the content of 2-MAG is relatively low, which is 15.27%; in FIG. 2B, the content of 2-MAG is 21.87%, see Table 7 for summary. It is indicated that in the ester exchange alcoholysis reaction, the raw materials with peroxide value reduction treatment have a significantly increase in the persistence of enzyme activity, which is specifically embodied in the fact that after the lipase is used for 10 times, the content of the generated 2-MAG is higher than that of the lipase used when the raw material is not subjected to deodorization treatment.

TABLE 7
Influence of deodorization treatment and non-deodorization
treatment of raw materials on 2-monoglyceride
content in crude ester exchange products

| | Non-deodorization | Deodorization |
|---|---|---|
| | treatment of raw | treatment of raw |
| Lipid | materials (FIG. 2a) | materials (FIG. 2b) |
| 2-monoglyceride content (%) | 15.27 | 21.87 |

Note:
the enzyme has been used for 10 times.

### Comparative Example 3: (Compared With Example 4)

Different from example 4, raw materials used in comparative example 3 are not subjected to peroxide value reduction treatment. Experimental conditions and steps for synthesizing the structured lipid through hydrolysis are identical to example 4.

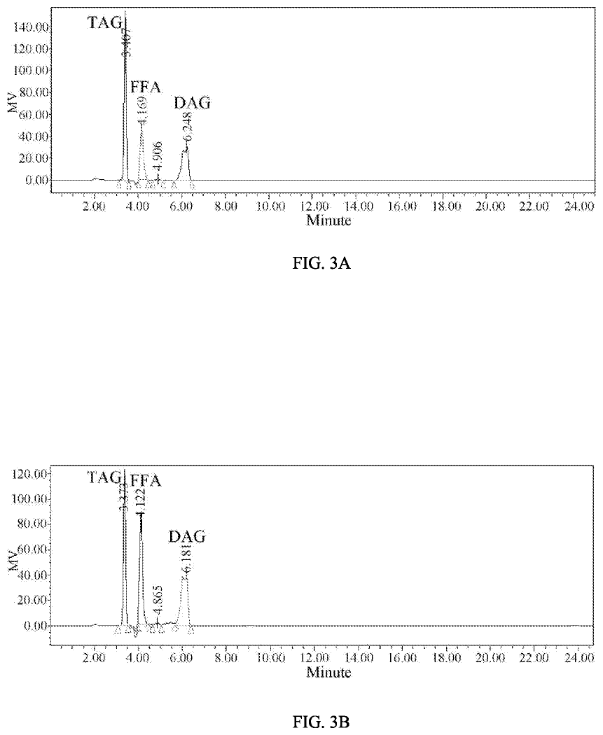

The enzyme retrieved after the raw materials treated with adsorption under the optimal conditions (2% silica gel, room temperature, and 20 min) in example 4 and hydrolyzed is taken and used for synthesis reaction for 10 times, and the content of 2-MAG obtained from 10 times reaction is compared with the content of 2-MAG obtained from 10 times reaction using the enzyme of comparative example 3, see FIGS. 3A-3B. FIG. 3A is a liquid chromatogram of the raw materials with no peroxide value reduction treatment, and FIG. 3B is a liquid chromatogram of the raw materials subjected to absorption treatment. In FIG. 3A, the content of DAG is 27.74%; in FIG. 3B, the content of DAG is 33.78%, see Table 8 for summary. It is indicated that in the hydrolysis reaction, the raw materials subjected to peroxide value reduction treatment have a significantly increase in the persistence of the enzyme activity, which is specifically embodied in the fact that after the lipase is used for 10 times, the content of the generated DAG is higher than that of the lipase used when the raw material is not subjected to adsorption treatment.

TABLE 8
Influence of adsorption treatment and non-adsorption treatment of
raw materials on diglyceride content in crude hydrolysis products

| | Non-adsorption | |
|---|---|---|
| | treatment of raw | Adsorption treatment of |
| Lipid | materials (FIG. 3a) | raw materials (FIG. 3b) |
| Diglyceride content (%) | 27.74 | 33.78 |

Note:
the enzyme has been used for 10 times.

It should be noted that the above examples are only for illustrating the technical solution of the present disclosure but not limited thereto. Although the present disclosure is described in detail by referring to preferred embodiments, persons of ordinary skill in the art should understand that modifications or equivalent substitutions can be made to the technical solution of the present disclosure, and should be included within the claim scope of the present disclosure without departing from the spirit and scope of the technical solution of the present disclosure.

1. A method for increasing the use efficiency of a lipase during an enzymatic reaction comprises:
conducting a peroxide value reduction treatment on a substrate used for synthesizing a structured lipid; and
subjecting the substrate to undergo a catalytic reaction with the lipase to synthesize the structured lipid;
wherein the peroxide value reduction treatment comprises a distillation method and an adsorption method; wherein the distillation method comprises a deodorization and a molecular distillation.

2. The method according to claim 1, wherein the temperature of the deodorization is 220° C.-270° C., and the time of the deodorization is 30 mins-150 mins.

3. The method according to claim 2, wherein the temperature of the deodorization is 240° C.-270° C.

4. The method according to claim 2, wherein the molecular distillation has a evaporation surface temperature of 200° C.-290° C.

5. The method according to claim 1, wherein the adsorption method uses an adsorbent comprising silica gel, activated clay, activated carbon, zeolite, diatomite and attapulgite.

6. The method according to claim 5, wherein the addition amount of the adsorbent is 0.5%-6% of the weight of the substrate.

7. The method according to claim 6, wherein the adsorption time of the adsorption method is less than 60 mins.

8. The method according to claim 1, wherein the lipase comprises any lipase used for catalyzing hydrolysis reaction, ester exchange reaction or esterification reaction.

9. The method according to claim 1, wherein the structured lipid comprises a human milk fat substitute, a cocoa butter substitute, a medium and long carbon-chain fatty acid structured lipid and a triglyceride containing a polyunsaturated fatty acid.

10. A use of the preparation method according to claim 1 in the fields of structured lipid preparation and food.
